(19) United States
(12) Patent Application Publication
(10) Pub. No.: US 2022/0411293 A1
(43) Pub. Date: Dec. 29, 2022

(54) MAGNET BAND FOR PRODUCTION OF MAGNETIZED WATER

(71) Applicant: BIOMAGNETEC CO., LTD., Seoul (KR)

(72) Inventor: June Kyoo KIM, Gyeonggi-do (KR)

(21) Appl. No.: 17/787,632

(22) PCT Filed: Dec. 17, 2020
(86) PCT No.: PCT/KR2020/018501

§ 371 Date: Jun. 21, 2022

(30) Foreign Application Priority Data
Dec. 18, 2019 (KR) 10-2019-0169963

(51) Int. Cl. C02F 1/48; A61N 2/06

(52) U.S. Cl. CPC C02F 1/48; A61N 2/06

(57) ABSTRACT

A magnet band for production of magnetized water according to an embodiment of the present disclosure is easy to carry and convenient to use so that a user can easily magnetically activate and drink a beverage, and has a structure with reduced production cost so that many people can inexpensively and widely use the magnet band.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/018501, filed Dec. 17, 2020, which claims priority to the benefit of Korean Patent Application No. 10-2019-0169963 filed in the Korean Intellectual Property Office on Dec. 18, 2019, the entire contents of which are incorporated herein by reference.

### BACKGROUND

### 1. Technical Field

The present disclosure relates to a magnet band for producing magnetized water, particularly, a magnet band for producing magnetized water, the magnet band enabling a user to simply magnetize and take a drink because it is easy to carry and use, and being able to be used by many people at a low cost because the manufacturing cost is reduced.

### 2. Background Art

Water is not only a substance that is necessary for human being to maintain life and carry on daily life, but also an important fundamental resource that makes social activities, such as economics, industry, and culture, harmonious. Accordingly, technologies of securing and supplying water of good quality have been developed in the history, and the demand for the quality of water is increasing with variations of social conditions such as industrial development and advancement of life.

However, air pollution is recently getting extremely severe with industrial development, and wastes that are byproducts due to industrial development are intactly buried in many cases, leading to the environment being contaminated. As a result, it is impossible to intactly drink or use underground water as water for industrial use due to contamination of the sources of drinking water, so water is purified at an intake station and then supplied to common home or various industrial facilities.

Even water purified at an intake station has to pass through water supply pipelines to be supplied to common home or industrial facilities. However, since most water supply pipelines are old, rust is generated or foreign substances are accumulated on the inner surfaces of the pipes, so there is a problem that the water that is finally supplied common home is not clean.

Further, since chemicals such as chloride are added to water for purification, there is a trouble that it is required to leave water for a predetermined time in order to evaporate the chloride in the water or sink foreign substances in the water and then use the water for drinking.

Since tap water that is supplied to common home or water for industrial use that is supplied to various industrial facilities has a huge complex structure due to hydrogen bond of many water molecules, activity of water itself is very low, so there is a problem that the purification effect, adjustment effect, extraction effect, reduction effect, etc. that are intrinsic to water are not achieved well.

Further, since harmful microorganisms are mixed into water that is supplied to common home and various industrial facilities while the water passes through water supply pipelines, there is a problem that not only clarity of the water is very low, but an offensive odor is generated.

In order to solve these problems, recently, a water purifier is mounted on a faucet to purify water that is supplied in common home, and supplied water is intactly used as water for industrial use without a specific purification process or is purified through a separate filter and then used at various industrial facilities. However, there is a problem that such purifiers or filters only filter out foreign substances in water and cannot activate the water that is supplied.

Magnetism generators are installed in pipelines, through which water flows, to activate the water using magnetism, but existing products cannot be carried because they are manufactured to be installed at fixed places such as a pipeline, so there is a problem that activated water can be used and drunk only at limited places.

### SUMMARY

The present disclosure has been made in an effort to solve the problems described above, and an objective of the present disclosure is to provide a magnetized water generator that can be simply carried and used anytime anywhere by anybody because the volume is small.

Another objective of the present disclosure is to provide a magnetized water generator that can be simply installed at various places without limitation in installation place and installation type such as a pipe and container.

Another objective of the present disclosure is to provide a magnetized water generator that can be carried and used by many people at a low cost because the structure is simplified to be able to minimize the manufacturing cost.

In order to achieve the objectives, a magnet band according to the present disclosure includes: a band (101) formed in a band shape; Velcro tapes (102) formed at both sides of the band (101), respectively, and fixing the band (101) when the band (101) is wound on an installation object; and a magnetism generator (110) fastened to the band (101) and generating magnetism.

Covers (103) made of leather, leather replacement, etc. are formed on opposite sides to the Velcro tapes (102) of the band (101).

The magnetism generator (110) has a housing (111) made of leather, leather replacement, etc. such that a space is formed therein, and has a magnet (113) disposed in the space of the housing (111), and the housing (111) is fastened to the band (101).

A cover (112) made of leather, leather replacement, etc. is fastened to an open top of the housing (111).

The magnet of the magnetism generator (110) is installed so that N-pole faces an installation object.

Since the magnet band of the present disclosure has a body formed in a band shape that is small in volume and flexible, it is possible to simply roll and carry the magnet band and conveniently use the magnet band anytime anywhere without limitation in place and time.

Further, it is possible to activate water flowing through a pipe by winding the magnet band on the pipe, and it is also possible to simply activate and drink water in containers by winding the magnet band on a cup, a tumbler, or the like, so it is possible to install and activate water at various places.

Further, since the manufacturing cost can be minimized due to a simple structure, many people can carry and use the magnetized water generator at a low cost.

### BRIEF DESCRIPTION OF THE DRAWINGS

FIG. 1 is a plan view showing a magnet band according to the present disclosure.

FIG. 2 is a top perspective view showing the magnet band according to the present disclosure.

FIG. 3 is a bottom perspective view showing the magnet band according to the present disclosure.

FIG. 4 is a vertical cross-sectional view showing the magnet band according to the present disclosure.

FIG. 5 is a view showing the state in which the magnet band according to the present disclosure is wound on a container to be used.

FIG. 6 is a view showing the state in which the magnet band according to the present disclosure is wound on a wrist to be used.

### DETAILED DESCRIPTION

Hereafter, the present disclosure will be described with reference to preferred embodiments of the present disclosure and the accompanying drawings under the assumption that the same reference numerals indicate same components.

When any one component “includes” another component in the detailed description or claims of the present disclosure, it is not construed as including only the corresponding component, but should be understood that other components may be further included unless stated otherwise.

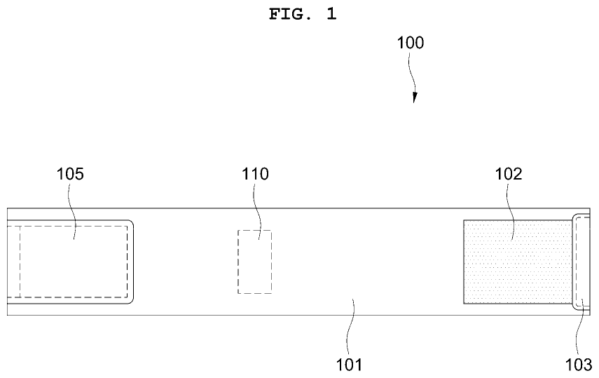

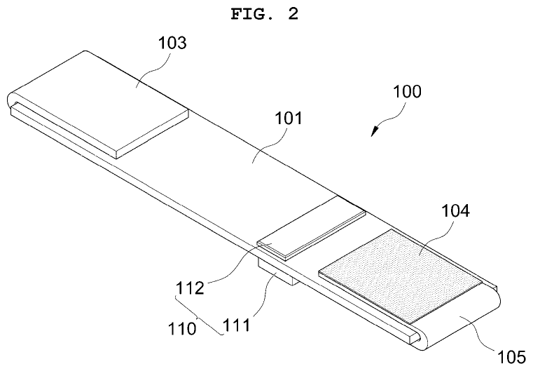

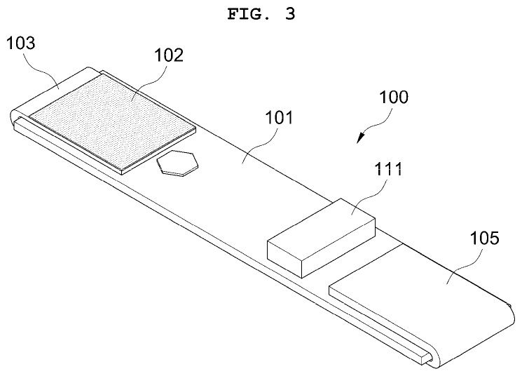

A magnet band for producing magnetized water according to the present disclosure, as shown in FIGS. 1 to 4, includes a band-shaped band 101 and Velcro tapes 102 and 104 at both ends of the band 101.

A flexible material that can be wound on a pipe, a container, etc. is used for the band 101, and it is preferable that the band 101 is made of an elastic material.

The magnet band 100 of the present disclosure is configured to be wound on an installation object such as a pipe or a container. By configuring the band 101 as an elastic band, the band 101 is wound on an installation object and fastened and fixed by the Velcro tapes 102 and 104 at both ends of the band 101.

Further, the elastic band 101 elastically presses the installation object such that a magnetism generator 110 to be described below is in close contact with the installation object.

The Velcro tapes 102 and 104 are formed on the outer side of an end of the band 101 and on the inner side of the other end of the band 101, respectively, as means for fixing the band 101 wound on an installation object. It is preferable to form covers 103 and 105 on the opposite sides to the Velcro tapes 102 and 104 of the band 101 using leather, leather replacement, etc.

Since the covers 103 and 105 are formed on the opposite sides to the Velcro tapes 102 and 104 of the band 101, the external appearance of the magnet band 100 is improved and the cover 105 functions as a grip when the Velcro tapes 102 and 104 are attached and detached, which makes attachment and detachment easy.

The band 101 has the magnetism generator 110.

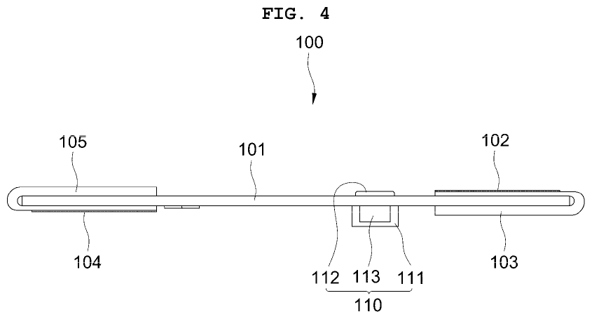

The magnetism generator 110, as shown in FIG. 4, has a housing 111 on a side of the band 101 and a magnet 113 inserted in the housing 111, and a cover 112 is fastened to the housing 111 and fastened to the band 101 by sewing, or the like.

It is preferable that the housing 111 and the cover 112 are made of leather, leather replacement, etc. that is the same material as that of the cover 103 of the Velcro tape 104.

It is preferable to make the product light by reducing the weight by using an Nd—Fe—B sintered magnet of magnets made of rare earth elements, which have the most intensive magnetism of the existing magnets, as the magnet 113.

As the magnet 113, an Nd—Fe—B sintered magnet, which is a magnetic material having intensive residual magnetic flux (Br) of 13,000 Gausses or higher, is disposed such that the N-pole that is advantageous for organisms faces an installation object.

For the dimensions of the magnet 113, it is preferable to use a magnet having magnetism that can activate water of 500 cc while the water is stirred for 30 seconds.

The principle that water is activated by magnetism uses electromagnetic induction by magnetic flux line found out by Faraday in 1831, that is, uses the fact that a current is generated by flow of electrons in a conductor moving perpendicular to magnetic flux lines, so ionization in which water molecules easily has electrical properties further progresses and water is decomposed into hydrogen ions (H⁺) and hydroxide ions (OH⁻) and the fact that since water molecules cannot independently exist, they are repeatedly produced, disintegrated, and produced again during a very short time so that the water molecule clusters having a size increased by contamination returns to the original small size due to magnetic flux lines.

Natural water is also contaminated due to microplastic or endocrine-disrupting substances when it is kept in a plastic bottle and carried. Tap water that is used at cities, etc. are also contaminated by many chemical substances stuck between clusters, such as chlorine, calcium hypochlorite, a detergent, agricultural chemicals, antiseptics, ammonia, and the like. Accordingly, the clusters of water molecule is very large in size so that when frozen, the crystalline structure is formed in a polygon rather than in a hexagon like the crystalline of snow, or is not even formed in any polygonal shapes to have large clusters. However, when the water is activated by magnetism, the increased size of the water molecule clusters returns to the initial small size.

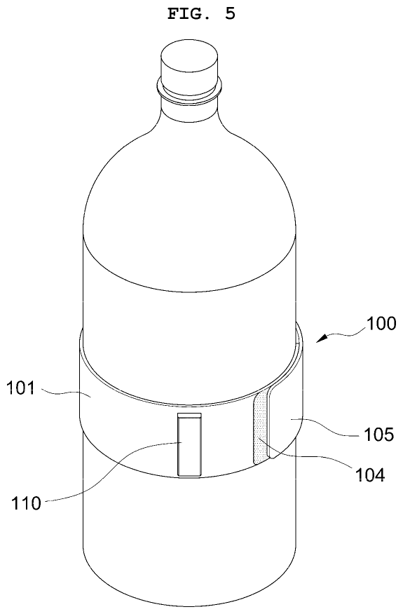

The present disclosure having the configuration described above, as shown in FIG. 5, is wound on a container with a drink therein and fixed by the Velcro tape 104, so the drink in the container is activated by the magnetism generated by the magnetism generator 110.

Further, though not shown in the figures, the magnet band 100 of the present disclosure may be wound and fixed on a pipe through which water flows so that the water flowing through the pipe is activated by intensive magnetism generated by the magnetism generator 110.

Further, it is possible to achieve a magnetic therapy effect by wearing the magnet band 100 of the present disclosure around a human body.

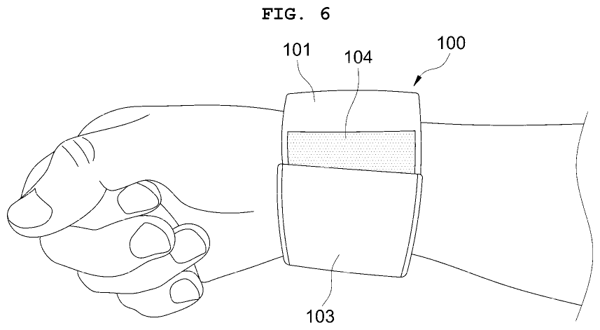

As shown in FIG. 6, by wearing the magnet band 100 of the present disclosure on the part with a pain when a pain is generated at a wrist or an ankle, it is possible to achieve an effect like magnetic pain relief patches or magnetic stomach bands that are generally used to remove pain.

The spirit of the present disclosure was described above with reference to an embodiment.

It is apparent that the embodiment described above may be changed and modified in various ways from the above description by those skilled in the art.

Further, although not definitely shown or described, it is apparent that various modifications may be accomplished including the spirit of the present disclosure from the above description by those skilled in the art, which is included in the scope of the present disclosure.

The embodiment described above with reference to the accompanying drawings was provided to describe the present disclosure and the scope of the present disclosure is not limited thereto.

1. A magnet band, comprising:
a band formed in a band shape;
velcro tapes formed at both sides of the band, respectively, and fixing the band when the band is wound on an installation object; and
a magnetism generator fastened to the band and configured to generate magnetism.

2. The magnet band of claim 1, wherein covers made of leather, leather replacement, etc. are formed on opposite sides to the velcro tapes of the band.

3. The magnet band of claim 1, wherein the magnetism generator has a housing made of at least one of leather and leather replacement such that a space is formed therein and has a magnet disposed in the space of the housing, and the housing is fastened to the band.

4. The magnet band of claim 3, wherein a cover made of at least one of leather and leather replacement is fastened to an open top of the housing.

5. The magnet band of claim 1, wherein the magnet of the magnetism generator is installed so that N-pole faces an installation object.
